(19) United States
(12) Patent Application Publication
(10) Pub. No.: US 2011/0237950 A1
(43) Pub. Date: Sep. 29, 2011

(54) METHOD AND APPARATUS FOR MULTI-BEAM BEAMFORMER BASED ON REAL-TIME CALCULATION OF TIME DELAY AND PIPELINE DESIGN

(75) Inventor: Guohai MENG, Shenzhen (CN)

(73) Assignee: SHENZHEN LANDWIND INDUSTRY CO., LTD., SHENZHEN (CN)

(21) Appl. No.: 12/793,698

(22) Filed: Jun. 4, 2010

(30) Foreign Application Priority Data
Mar. 23, 2010 (CN) 201010133657.7

(51) Int. Cl. A61B 8/14

(52) U.S. Cl. 600/447

(57) ABSTRACT

A multi-beamforming method based on real-time calculation of delay parameter and pipeline technique and an apparatus there of are disclosed. The system separates the parameters that should be calculated in real time from the parameters that does not require real-time calculation, and separates the parameters related to the beam sequencing number and the parameters that are independent of the beam sequencing number, and provides a real-time delay calculation unit that is adapted to different types of probe by means of simple switching. The calculation unit utilizes the pipeline design, the delay parameters of M number of beams are calculated in the calculation unit in pipeline manner, and then the memory of the same channel echo data is read, so that the delay of the beams is realized. The consumption of the FPGA resource is greatly reduced. The present invention enables high delay precision through direct calculation. In order to reduce the occupation of the hardware resource, the present invention uses the pipeline design to allow M number of beams to share the delay parameter calculation unit. The occupation of the hardware resource is greatly reduced accordingly.

### BACKGROUND OF THE INVENTION

This invention relates to an ultrasound imaging system using linear probes, convex probes and phased array probes, in particular to a multi-beam beamforming system based on real-time calculation of time delay and pipeline design.

The quality of ultrasound imaging has been greatly improved in recent decades, owing to many advanced techniques in radar and the digital signal processing and image processing technique being used. However, many of them are always at the cost of reducing frame rate. For example, in the spatial compound imaging, it is necessary to synthesize images of multi-frame scanned from different angles to obtain the final image; phase-inversion tissue harmonic technique requires that two frame images obtained from different transmitting pulse polarity are superposed to obtain the tissue harmonic image; and synthetic aperture focusing technique requires the superposition of multi-frame images to realize the point-by-point focusing in both transmission and receiving. In modern color Doppler ultrasound system, a very high transmitting pulse repeat frequency is required to extract the blood flow signal, and therefore the time required to obtain the 2D B-mode ultrasound image is reduced greatly, and the frame rate is also reduced significantly. For fast imaging being required for observing moving object in clinical practice, the great reduction of frame rate will limit its use in cardiopathy diognosis.

Multi-beam beamforming technique makes up the reduction of frame rate of B-mode ultrasound image caused by the above-described techniques. Comparing with the normal way of scanning, the multi-beam beamforming technique may generate M number of scan lines (normally, M is in a range of 2 to 16) simultaneously during one transmitting process, and thus the frame rate is improved by M times. However, it is a difficult technical problem to implement the multi-beam technique. In single-beam beamformer, in order that it can be realized technically and to reduce the cost, the focus parameters are stored in advance to realize the dynamic focusing in receiving. In this way, a huge quantity memory are required to implement multibeam beamformer. Meanwhile, the demand with respect to the FPGA resource is increased by several times such that it is impossible to be realized. In the present invention, the time delay of every channels required for the beamforming is calculated in real time by means of pipeline design, and the focusing parameters needed to be stored are only the parameters related to the probe and the direction of scan line. In this way, for each scan, if four beams are formed each time, only about 160 bytes memory space is required in order to store all the focusing parameters. Considering 64 weighted values of variable weight points are set along the scan line, and each weighted value is one byte, it requires a memory capacity of 1 k bytes for the weighting parameters of 16 channels, and in this manner, for the condition that an echo is synthesized into four wave beams, parameters needed to be stored only occupy 1184 bytes. The data needed to be stored for 256 scan lines is 296 k bytes.

For the reason that the multi-beam technique is significant for improving the frame rate and the quality of the B-mode ultrasound image, the multi-beam technique is given high attention in the ultrasound imaging field, and many technical solutions and patents are developed accordingly. Among the patents related to the beamformer, two types may be classified according to whether the focusing delay parameter is pre-calculated or calculated in real time. In the early single beam beamformer, the delay parameter is usually pre-calculated and then stored. In order to reduce the space occupied in the memory by the parameters, the combination of initial value and increment is normally used. The initial value of delay is normally coarse time delay (that is, the integer sampling clock cycle is taken as the unit of the time delay), and the time delay that its precision is higher than one sampling clock cycle is represented by the increment “0” and “1”, and the unit of fine time delay is normally ¼ to ⅛ of the sampling cycle. In this way, the time delay parameter at the focus of different depth of each channel is in fact one bit data flow. However, for the quantity of focus that realizes the dynamic focusing may reach up to one thousand to even several thousands, the great memory capacity is still needed. In order to reduce the demand to the memory capacity and the occupation of FPGA resource, real-time calculation and Time Division Multiplexing (TDM) technique are widely used for the time delay parameter in the multi-beam technique.

U.S. Pat. No. 5,905,692 (with Publication Date of May 18, 1999, and corresponding Chinese Patent No. 98812777.6 with Publication Date of Feb. 7, 2001) discloses a way of implementing multi-beams through the use of Time Division Multiplexing. However, this patent does not disclose how to generate time delay parameters.

U.S. Pat. No. 6,123,671 (with Publication Date of Dec. 31, 1998) discloses an apparatus for calculation of beamforming time delays and apodization values in real-time based on CORDIC algorithm. In this apparatus, the calculation of the time delay is based on the element position coordinates x, z and the focus position coordinates x, z. Therefore, it is adapted to the probe in any shape. However, for the reason that the element position coordinates and the focus position coordinates should be stored simultaneously, a large storage amount is also needed, and if each focus position coordinate is calculated, the calculation would be too complicated and too much FPGA resource would be occupied. Furthermore, all 16 channels in the same chip are subject to this delay calculation system by the use of Time Division Multiplexing, and thus only a calculation frequency of 2.5 MHz in each channel can be achieved. This is relative low for some specific applications.

U.S. Pat. No. 7,508,737B1 (with Publication Date of Mar. 24, 2009) discloses a method in which a plurality of receive channels use the same delay control by the use of Time Division Multiplexing, a low-pass filter is used to realize the interpolation operation, and the interpolation can be performed after summing signals of all channels, and therefore the hardware complexity is reduced. However, this patent does not disclose the method for implementing the time delay parameters.

U.S. Pat. No. 5,469,851 (with Publication Date of Nov. 28, 1995) discloses a time multiplexed digital ultrasound beamformer. In this patent, the delay output is divided into two groups, i.e. the primary delay and the neighbor delay. The neighbor delay may be calculated from the primary delay, and thus it is simplified. The time delay is realized by using the dual port RAM, the write counter, and the read counter. The read counter is stalled according to the delay control to realize a change in delay. The time delay includes the coarse delay and the fine delay. The fine delay is realized through the use of a sixth order low pass filter by selecting different filter coefficient. The calculation of its delay parameter is given in another U.S. Pat. No. 5,522,391 in which each focus delay is calculated by using the recursive algorithm. The biggest problem of the recursive algorithm is that accumulated error may be induced.

Chinese Patent No. 200610021344.6 (with Publication Date of Jan. 2, 2008) and Chinese Patent No. 200610168851.2 (with Publication Date of Jun. 11, 2008) disclose a method for calculating the focusing parameter in real time and a system thereof, wherein the algorithm of delay parameter includes the feedback from the final stage towards the frontend, and therefore the calculation of delay parameter can not realize the pipeline operation and the time division multiplexing.

As for the calculation of delay parameter in real time, there are mainly three types of algorithm. The first type is direct calculation, the sound path is calculated mainly according to the array element coordinates and the focus coordinates to educe the delay parameter. The second type is approximation algorithm, and this is to avoid the square root calculation in the direct calculation, and in order to reduce the amount of calculation, first order approximation or second order approximation is normally used. The third type is recursive algorithm, and a next focus delay is calculated from a known previous focus delay. Among these three algorithms, more hardware resources are needed and the requirement to the hardware speed is relative high in the first type. However, with the development of the FPGA, this problem becomes less important. As for the second type, the precision is so limited for the approximation calculation and may not meet the requirement of the focusing precision. The third type has the minimum amount of calculation, but may bring accumulated error and with the increase of depth of the focus position, the accumulated error will seriously reduce the focusing precision.

At the present time, digital beamforming technique is widely used, and it mainly uses the delay parameters that are calculated and stored in advance to realize the time delays of signal in receive channels. This method is simple in structure, but it is required to add a relatively large RAM externally on the FPGA. For a single-beam system, this is a suitable solution. However, for a multi-beam system, it requires too much time for updating the RAM content due to the large capacity of the external RAM, so that it is not suitable for the multi-beam system. Therefore, in the multi-beam system, real-time calculation of the delay parameters is widely used. The real-time calculation of the delay parameters require more hardware, especially for the probe in different shape, the design solution will become very complicated.

FIG. 1 is a block diagram of a typical digital beamforming ultrasound imaging system. Under the control of the control unit 80, the transmitter 30 generates a group of pulses with focusing time delay to the transducer array 10. The transducer array 10 converts the electrical pulse signal into the ultrasound pulse with different phase for the array elements. The ultrasound pulse converges in the forward direction according to the predetermined phase to form the focusing beam. The focusing beam transmitted by the transducer array 10 is reflected by the human tissue and then converted into the receiving electrical signal by the transducer array 10. Normally, the transducer array has 128 or more transducer array elements, while the physical channels for receiving are normally fewer than the quantity of the transducer array elements. The switch unit 20 performs switching selected elements to the receiving channels. The group of receiving signals selected pass through the transmit/receive switch in the switch unit 20 to suppress the entry of the transmitting pulse so as to prevent the blocking of the analog channel, and then is transferred to the analog frontend and the ADC module 21. In the frontend and ADC module 21, the echo signals received is applied with pre-amplification, TGC (time gain compensation) amplification and finally ADC (Analog-to-Digital) conversion. The digitized ultrasound echo signal is transmitted into the beamforming unit 40 that is used for dynamically delaying each echo signal, and the delayed signal is performed with summing operation, and a group of echo is formed into a wave beam which we call a scan signal. For the dynamic focusing, the beamformer unit 40 will calculate the time delay amount for each sample point of receiving signal. Therefore, the beamforming unit requires external RAM to store the focusing parameters. After the beamformer, the scan signal passes through the signal processing and image processing unit 50 and then the digital scan conversion unit 60 to form a raster image. Finally, under the control of the controller, the ultrasound image signal is transmitted to a computer for further processing and display by the bus controller and the computer bus.

FIGS. 2 and 3 illustrate the principle block diagram of the beamformer unit, wherein FIG. 2 is a delay circuit of one channel, and FIG. 3 is a summing circuit of N-channel echo signals. Under the control of write control unit 42, the write address counter 43 generates a linear write address for the ith channel echo signal after passing through the analog frontend and the ADC conversion. The echo signal i is continuously written into the dual port RAM 41. An initial count value is set into the read address counter 46 at the beginning, which we call coarse delay. The coarse delay represents the offset of the first read echo signal data with respect to the write address, and is also the integer portion of the ith channel delay amount that is represented by sampling clock cycle. The fraction portion of the delay amount of the ith echo signal is also called as fine delay, that is, the portion small than a sampling clock cycle is done by the interpolation circuit 45. It uses the read data corresponding to the integer portion and the next data to obtain the intermediate data of them by means of interpolation. The interpolation coefficient is given by the read controller 47. Every time that the accumulated fine delay reaches a whole delay unit, the read address counter will stop counting once, which we call stalling. The stalling is controlled by the read controller 47 according to the delay parameters generated by the delay parameter generator 44. Because the delay should be adjusted dynamically, the delay parameter generator 44 should obtain the data from the data bus dynamically, and distribute the delay parameter of each channel to the read controller and the write controller in each channel.

The echo signals after delay control are sent to the summing unit in FIG. 3 for the final beam-forming. The summing unit 48 is a multi-channel signal adder.

The beamforming unit described above is characterized in that the delay parameters are precalculated and stored, then these parameters are dynamically read during the ultrasound signal processing to control the write counter, the read counter and the interpolation operation unit directly.

### BRIEF SUMMARY OF THE INVENTION

It is an objective of the present invention to provide a method and an apparatus for calculating delay parameters in real time, which is adapted to be used in different probes of different shape and reduces the occupation of hardware resource. Considering different types of probe, the present invention provides a universal delay calculation apparatus. The apparatus separates the parameters that should be calculated in real time from the parameters that does not require real-time calculation, and separates the parameters related to the beam sequencing number and the parameters that are independent of the beam sequencing number, and provides a real-time delay calculation unit that is adapted to different types of probe by means of simple switching. The calculation unit utilizes the pipeline design, the delay parameters of M number of beams are calculated in the calculation unit in pipeline manner, and then the memory of the same echo signal data is read, so that the delay of the beams is realized. The consumption of the FPGA resource is greatly reduced. The present invention enables high delay precision through direct calculation. In order to reduce the occupation of the hardware resource, the present invention uses the pipeline design to allow M number of beams to share the delay parameter calculation unit. According to an aspect of the present invention, the parameters that require real-time calculation is separated from the parameters that do not need real-time calculation which is then designated as input parameters, so that the calculation amount is reduced. According to another aspect of the present invention, the parameters related to the beam sequencing number are separated from the parameters that are independent of the beam sequencing number, and the delay parameter unit can be used in the convex array probe, linear array probe and phased array probe by switching two switches.

To achieve the above objective, the technical solution of the present invention is as follows:

A multi-beamforming method based on real-time calculation of delay parameter and pipeline technique, wherein at least one multi-beamforming apparatus is used, each multi-beamforming apparatus receives ultrasound echo from a channel while generating corresponding delayed signals for M number of wave beams with different angles or scan positions. the method comprises the following steps:

A. each multi-beamforming apparatus processes one channel echo signal and generates delayed signals for M number of scan lines with different scan angles or positions;

B. the received echo signal is linearly written into a dual-port RAM by each multi-beamforming apparatus;

C. delay parameters for the M number of wave beams in the same channel are calculated according to array element parameters and focus position parameters of current channel, and converted into read addresses of the RAM;

D. within one write cycle, echo data after delay is read out from the RAM in turn according to the read addresses to generate M number of delayed signal outputs;

E. the M number of delayed signal outputs generated in the same channel are transmitted to an interpolation unit and a weighing unit in time division multiplexing manner for performing fine delay and apodization operation;

F. the first beams generated by all multi-beamforming apparatus are superposed to obtain a first synthesized beam,

the second beams generated by all multi-beamforming apparatus are superposed to obtain a second synthesized beam,
. . .

the Mth beams generated by all multi-beamforming apparatus are superposed to obtain a Mth synthesized beam,

the above operations are done in the same summing unit in pipeline and time division multiplexing manner;

G. the outputted M number of beam signals are transferred to the next stage summing unit in time division multiplexing manner for further summing operation between chips or performed with signal processing and image processing.

The delay parameter calculation formula in the step C is as follows:

the delay is expressed as:

$${τ}_{i} = \frac{( L - {𝓁}_{i} )}{c}$$

Wherein c is sound speed that is about 1540 m/s in the human tissue;

Convert the delay into unit of sampling period:

$${n}_{i} = \frac{( L - {𝓁}_{i} )}{c} {F}_{s}$$

For convex array probe:

lᵢ=\sqrt{(R+L)²+R²−2(R+L)R cos(θᵢ−θᵣ)}

For linear array probe:

lᵢ=\sqrt{L²+(xᵢ−xᵣ)²}

For phased array probe:

lᵢ=\sqrt{xᵢ²+L²−2xᵢL cos(90°−θᵣ)}

Wherein, τᵢ represents the time delay; nᵢ represents the time delay in sampling period unit; Fₛ represents the sampling frequency; θᵣ represents the angle between the receive line and the transmit line; polar coordinates of the ith array element is (θᵢ,R); the focus on the receive line is F, and the focal length is L; lᵢ is the path of ultrasound from the element i to the focus F; xᵢ is the coordinates of the ith array element; xᵣ is the coordinates of the scanning line;

Parameters are sorted into follows categories in present invention:

a) parameters related to the probe or the positions of element in the aperture, that is, the parameters may vary channel to channel, and it is represented by x here;

b) parameters related to the orientation angle of the scan line (for convex array probe, phased array probe) or the position of the scan line (for linear array probe), that is the parameters related to the beam, and it is represented by Y here;

c) parameters needed to be processed in real time, that is the focal length of the focus, and it is represented by L here;

The delay calculation unit is divided into two portions, common calculation portion and beam related calculation portion. Parameters X related to the channel and the real-time parameters L are sent to common calculation portion; based on the calculation output of the common calculation portion, the parameters related to the beam are calculated according to the inputted parameters Y's that are related to the beam.

The read frequency of the RAM is M times larger than the write frequency.

According to another aspect of the present invention, a multi-beamforming system based on real-time calculation of delay parameter and pipeline technique comprises an analog frontend that receives echo signal, the analog frontend is connected in sequence to an analog-to-digital conversion module, a DC cancellation module, a write control module, a RAM, an interpolation and weighting unit in pipeline manner and a channel summing unit in pipeline manner; the multi-beamforming system further comprises a real-time calculation unit of delay parameter in pipeline manner, the real-time calculation unit receives inputted parameters of M number of beams and calculates corresponding delay parameters that are then converted into read addresses of dual port RAM, the real-time calculation unit is connected in sequence to an address calculation unit and a read control module. The read control module is connected to the RAM and the interpolation and weighting unit in pipeline manner to control the reading out of the echo signal.

The real-time calculation unit of the delay parameter in pipeline manner consists of a common calculation portion A and a beam-related calculation portion B; the portion A processes the channel-related parameter X and the real-time parameter L; the portion B calculates the beam-related parameters according to the inputted beam-related parameters Y, based on the calculation output of the portion A.

The real-time calculation unit of delay parameter in pipeline manner has control signals C1 and C2, and the inputted parameters X, Y are set according to the type of probe, as shown in the following table:

| probe | X | Y | C1 | C2 |
|---|---|---|---|---|
| convex array | R | 2R cos(θᵢ − θᵣ) | 0 | 0 |
| linear array | 0 | (Xi − Xr)² | 1 | 1 |
| phased array | Xᵢ | 2Xᵢ cos(90° − θᵣ) | 0 | 1 |

In an preferred embodiment, the RAM is a dual port RAM, the read frequency of the dual port RAM is M times greater than the write frequency; in the multi-beamforming system where the number of beams is M, corresponding to each write data, the real-time address calculating unit will calculate M number of corresponding read addresses according to M number of outputs of the real-time delay parameter calculation unit and read M number of data according to these addresses from the dual port RAM.

In another preferred embodiment, the RAM is a tri-port RAM, the read frequency of the tri-port RAM is M times greater than the write frequency; in the multi-beamforming system where the number of beams is 2×M, corresponding to each write data, the real-time address calculating unit will calculate 2×M number of corresponding read addresses according to 2×M number of outputs of the real-time delay parameter calculation unit and read 2×M number of data respectively from two read ports of the tri-port RAM according to these addresses.

The method and apparatus of the present invention is operated completely in pipeline manner, and the delay parameter design unit can be used in the convex array probe, the linear array probe and the phased array probe through mode control and input parameter setting.

### BRIEF DESCRIPTION OF THE DRAWINGS

FIG. 1 is a schematic diagram of a known digitized beamforming ultrasound imaging system;

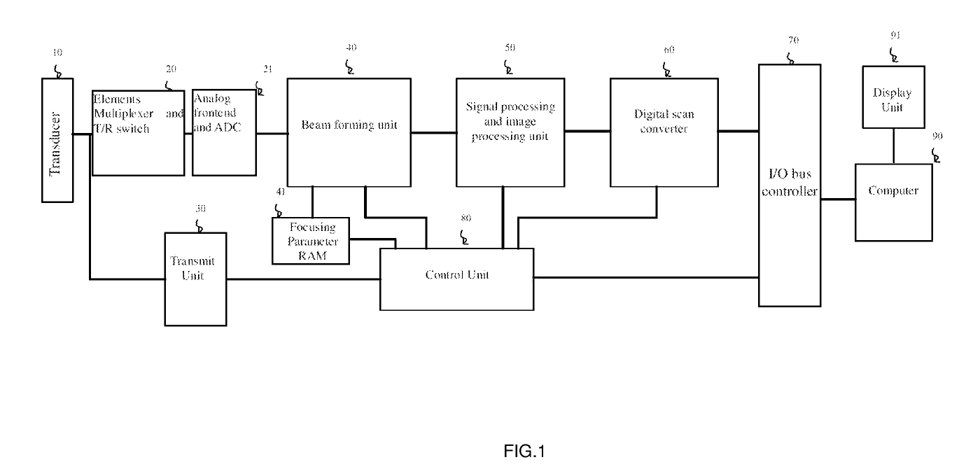

FIG. 2 is a schematic diagram of a dynamic delay circuit of an echo signal in the prior art;

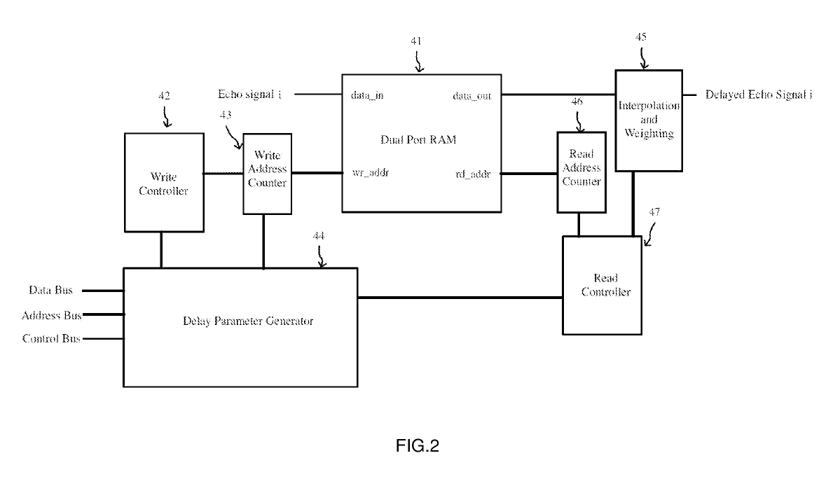

FIG. 3 is a schematic diagram showing the superposition of N number of echo signals after dynamic delay in the prior art;

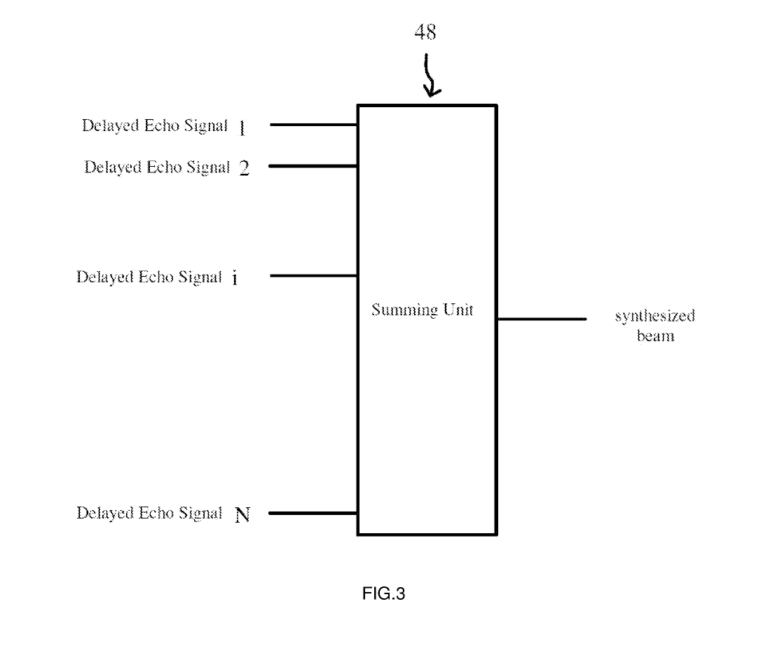

FIG. 4 is a schematic diagram showing the beamformer by the use of real-time calculation of delay parameters according to an embodiment of the present invention;

FIG. 5 is a schematic diagram showing the superposition of multi-beam beamformer according to an embodiment of the present invention;

FIG. 6 is a schematic diagram showing the multi-beam beamformer by the use of time division multiplexing according to an embodiment of the present invention;

FIG. 7 is a schematic geometrical diagram showing the delay calculation for the convex array probe according to an embodiment of the present invention;

FIG. 8 is a schematic geometrical diagram showing the delay calculation for the linear array probe according to an embodiment of the present invention;

FIG. 9 is a schematic geometrical diagram showing the delay calculation for the phased array probe according to an embodiment of the present invention;

FIG. 10 is a schematic diagram of a delay parameter calculation unit according to an embodiment of the present invention;

FIG. 11 is a schematic diagram of a four-beam delay parameter calculation unit in parallel arrangement according to an embodiment of the present invention;

FIG. 12 is a schematic diagram of a four-beam delay parameter calculation unit in pipeline arrangement according to an embodiment of the present invention;

FIG. 13 is a schematic diagram of a four-beam beamformer of the delay calculation unit according to an embodiment of the present invention;

FIG. 14 is a block diagram of a multi-beam system based on real-time delay parameter calculation, according to an embodiment of the present invention.

### DETAILED DESCRIPTION OF THE INVENTION

Many aspects of the present invention will be described in more detail in the following embodiments, with the accompanying drawings.

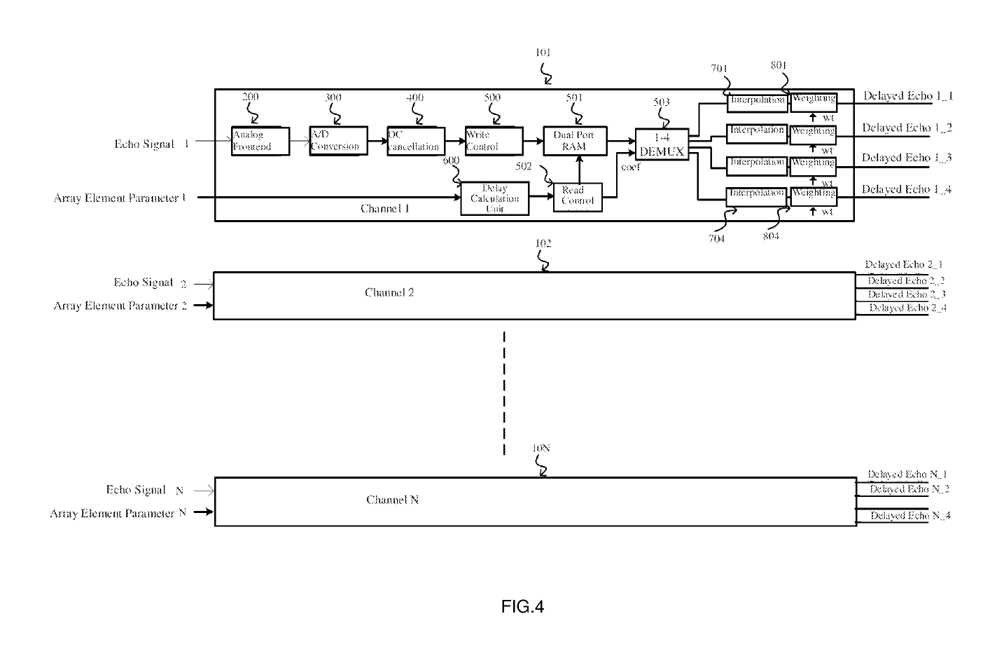

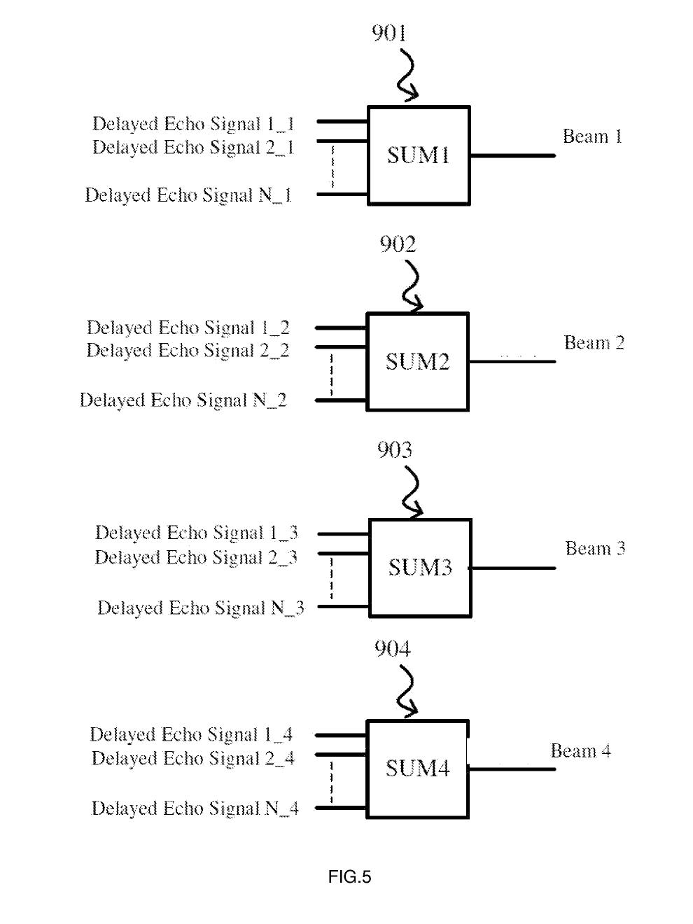

FIGS. 4 and 5 give examples of beamforming solution. In FIG. 4, the echo signal from the receive/transmit switch is transmitted to the analog frontend 200 and then the analog-to-digital converter 300 to be converted into digital signal. Then, the digital signal is transmitted to the DC cancellation unit 400 to filter the low frequency component and then be written into the dual port RAM 501 linearly under control by the control unit 500; the most important difference lies in that, the delay parameter calculation unit 600 is added in FIG. 4. The delay calculation unit calculates the delay amount in real time according to the element parameters corresponding to the current channel and the focus position parameter. As for the beamforming of M number of beams, the delay parameters of the M number of beams are calculated by the delay parameter calculation unit 600 in time division multiplexing manner, and then the echo data are read out from the dual port RAM 501 in turn based on the calculated addresses from 600 and 502 and can be transmitted to four interpolation units and weighting units through 1-4 DEMUX respectively for fine delay and apodization processing. Each echo signal after passing through the delay control circuit will generate M number of delay signal outputs. In FIG. 4, a typical example with four outputs is illustrated. Four outputs of each channel are respectively transmitted to the summing units 901, 902, 903, and 904 as shown in FIG. 5. The delayed signal i−1 represents the first beam of the ith channel, while the delayed signal i−2 represents the second beam of the ith channel, the rest may be deduced by analogy. The first beams of all channels are superposed to generate the first combined beam. Similarly, the second beams of all channels are superposed to generate the second combined beam. The rest may be deduced by analogy.

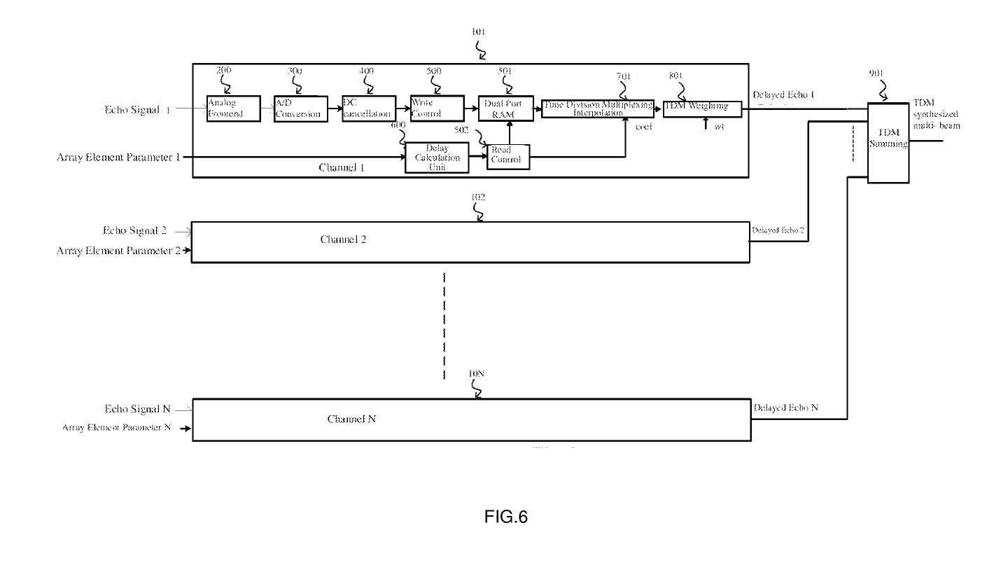

In FIGS. 4 and 5, the data from the dual port RAM is divided into four channels, which are merely used for illustrating the multi-beam concept. In a preferred embodiment, the time division multiplexing is used in the interpolation, weighting and summing units for all delayed echo signals of all beams so as to minimize the occupation of hardware resource, as shown in FIG. 6. FIG. 6 differs from FIG. 4 in that after the multi-beam delayed echo signals are read out from the dual port RAM 501, it is not divided into four channels for separate processing but transmitted to an interpolation unit 701 and a weighting unit 810, and the summing of the final beams is also done by a common summing unit 901.

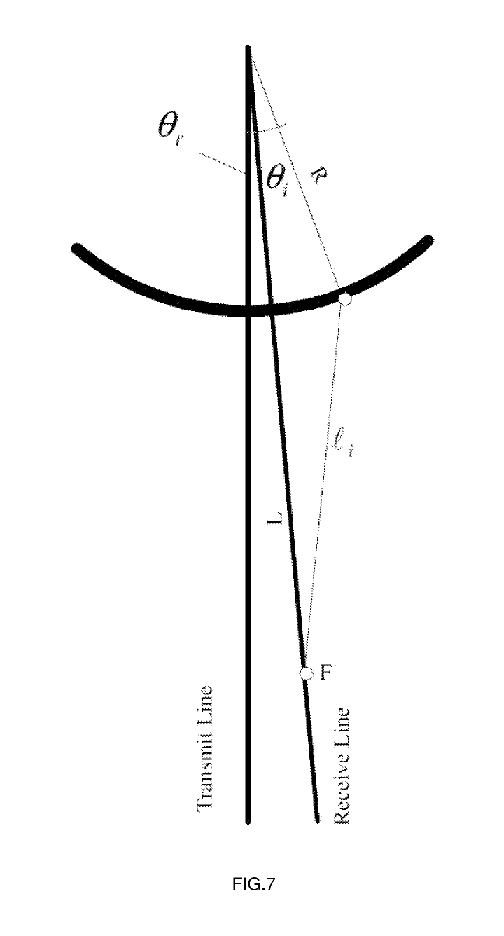

The real-time delay calculation unit is the most important point in the above solution. How to realize the real-time calculation of delay parameter will be explained hereinafter. FIG. 7 shows the delay calculation for the convex array probe. The transmit line (i.e. the central line of the transmit beam) is at the center of the aperture, the difference of the multi-beam receiving lies in that the receive line (i.e. the central line of the receive beam) does not necessarily coincide with the transmit line. Here, assuming that θᵣ is the angle between the receive line and the transmit line, the polar coordinates of the ith array element is (θᵢ, R). The focus on the receive line is F, and the focal length is L. According to cosine theorem, the following is the calculation formula of the sound path from the array element i to the focus F:

lᵢ=\sqrt{(R+L)²+R²−2(R+L)R cos(θᵢ−θᵣ)}

The delay is expressed as:

$${τ}_{i} = \frac{( L - {𝓁}_{i} )}{c}$$

Wherein, c is sound speed, which is about 1540 m/s in human tissue.

The delay is converted into sampling pulse unit:

$${n}_{i} = \frac{( L - {𝓁}_{i} )}{c} {F}_{s}$$

The delay calculation is mainly for calculating lᵢ. In the formula for calculating lᵢ, three types of parameter are contained:

1. Probe-related parameters: R, θᵢ {i=1:N; N is the quantity of array elements in the aperture}.

2. Scanning line parameter: θᵣ. In the convex array transducer and the phased array transducer, θᵣ is the scan angle.

3. Focus parameter: L. L may be written as i×ΔL. ΔL is focus spacing. This portion is the portion that requires real-time processing during beamfroming.

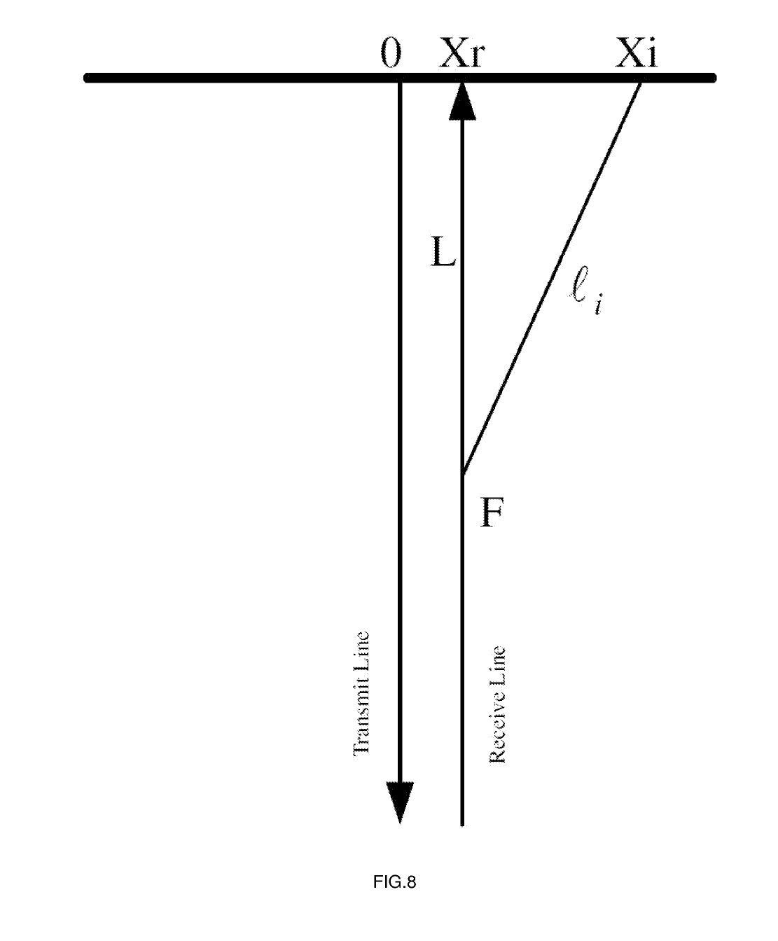

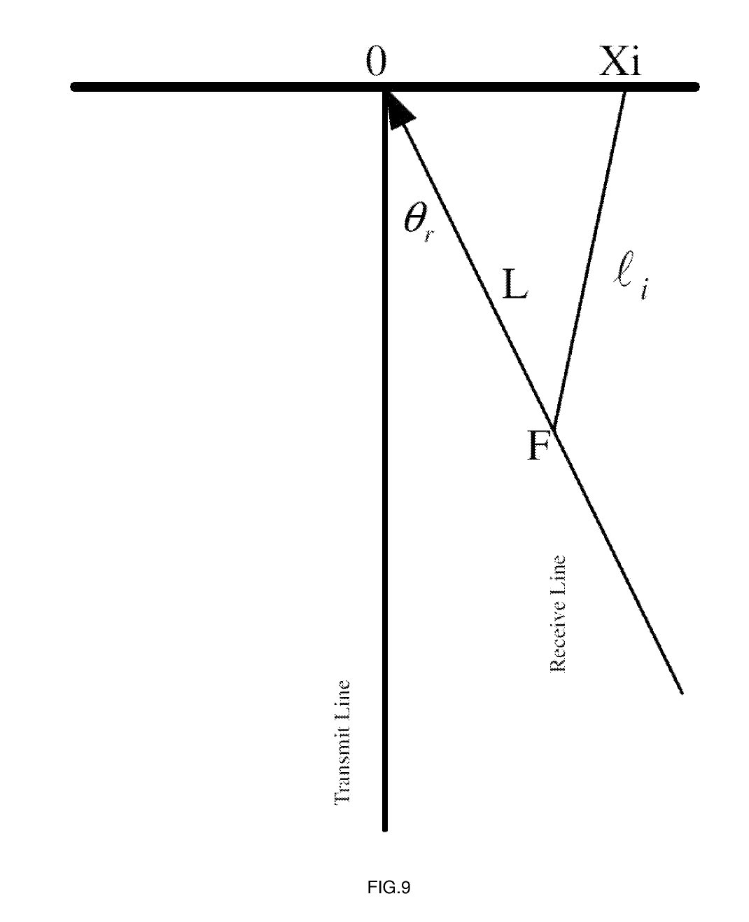

The delay calculations for the linear array probe and the phased array probe is respectively shown in FIG. 8 and FIG. 9. The calculation formula for sound path is as follows:

For Linear probe:

lᵢ=\sqrt{L²+(xᵢ−xᵣ)²}

For Phased array probe:

lᵢ=\sqrt{xᵢ²+L²−2xᵢL cos(90°−θᵣ)}

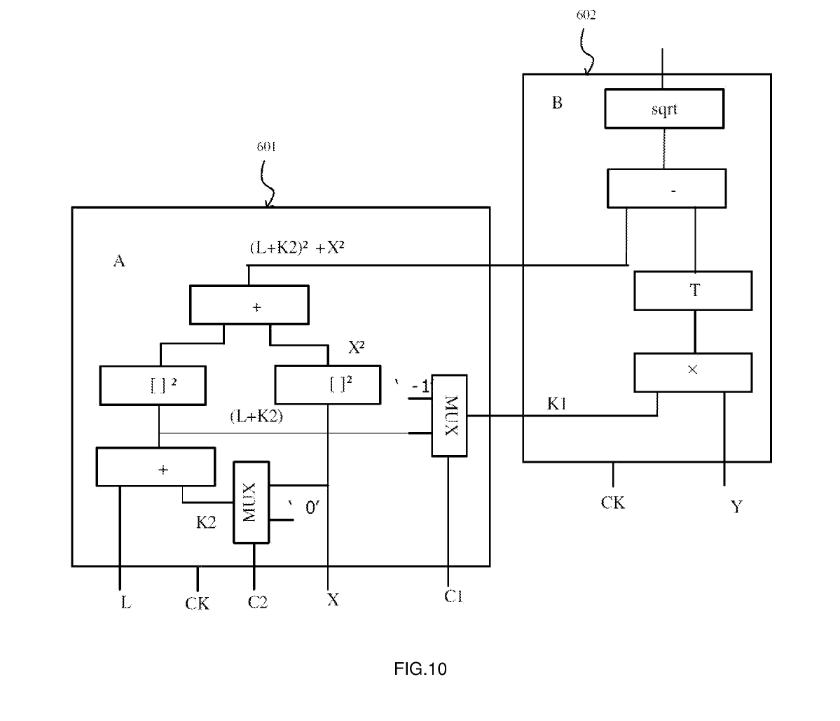

In the above two formulas, the parameter related to the array element is xᵢ, while the parameters related to the scan line are xᵣ and θᵣ, and the parameter related to the focus position is the focal length L. For minimizing the calculation amount, all parameters that are not related to the focal length L are calculated in advance and taken as input parameters. The parameters related to the scan line are parameters that distinguish the multi-beams, and should be inputted into the delay calculation unit in time division manner, so as to realize the time division multiplexing (TDM) of the delay calculation unit. In this way, a common delay calculation unit is show in FIG. 10. The inputted parameter L in FIG. 10 is still corresponding to the focal length. The inputted parameters X,Y are different according to different kinds of probe. Table 1 gives the parameters according to different kinds of probe.

TABLE 1
Inputted parameters of different probes

| probe | X | Y | C1 | C2 | remarks |
|---|---|---|---|---|---|
| convex array | R | 2R cos(θᵢ − θᵣ) | 0 | 0 | K1 = (L + K2), K2 = X |
| linear array | 0 | (Xi − Xr)² | 1 | 1 | K1 = −1, K2 = 0 |
| phased array | Xi | 2Xᵢ cos(90° − θᵣ) | 0 | 1 | K1 = (L + K2), K2 = 0 |

C1 and C2 are two control signals for selecting the two 2-1 multiplexers in FIG. 10, and the output values K1 and K2 represent different variable while the state of multiplexer is different, as shown in table 1. This changes the circuit form such that it may be used for calculating the delay parameters for the convex array probe, the linear array probe, and the phased array probe. “T” in FIG. 10 represents the delay of a clock cycle. The delay is added for meeting the requirement of pipeline operation. Therefore, no matter what setting is used, the solution in FIG. 10 can be operated in pipeline manner so as to support the time division multiplexing of different beams or different channels. The unit of all the inputs in table 1 is in geometric unit, and thus it should be quantified into sample period unit. The particular arithmetic is that multiplying all inputs by the quantification factor Fₛ/c. Fₛ is the sampling frequency, and c is the sound speed. In order to ensure the calculation precision, all values are retained with three binary digits in fraction.

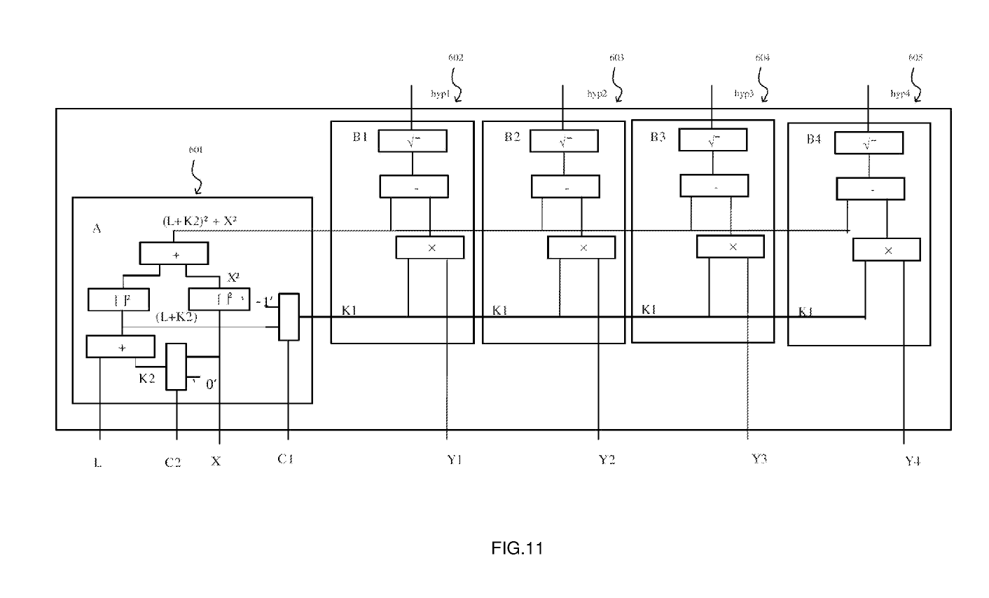

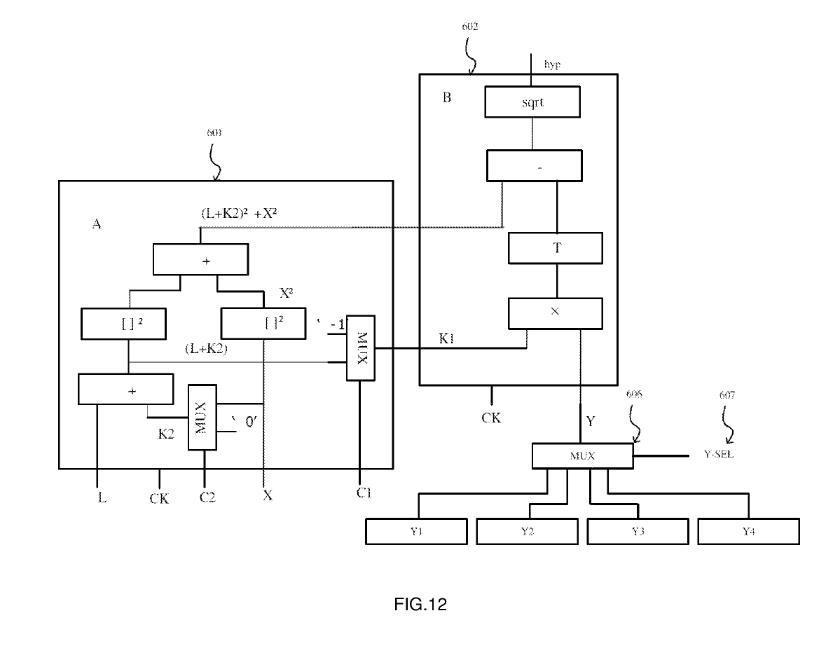

As shown in FIG. 10, the delay parameter calculation unit is divided into two portions A and B, the portion A 601 is the common calculation portion of multi-beams of the same channel, while the portion B 602 is a calculation portion corresponding to particular beam. Only the inputted parameter Y is related to the orientation angle of the echo (in the case of the convex array or phased array scanning) or echo position (in the case of the linear scanning). Therefore, when considering the pipeline operation, only Y is required to be switched among different beam. FIG. 11 and FIG. 12 illustrate respectively the four-beam delay parameter calculation in parallel manner and in pipeline manner.

In FIG. 11, only one common portion 601 is shown, and the delay parameter outputs of four beams are respectively corresponding to the module 602, 603, 604 and 605. The parameters Y1, Y2, Y3 and Y4 related to the four beam orientation angles and are respectively inputted into the module 601, 602, 603 and 604.

In FIG. 12, a common portion 601 and a beam calculation portion 602 are shown, and the beam-related parameters Y1, Y2, Y3 and Y4 are inputted into the module 602 via the multiplexer 606. The multiplexer selects the switching frequency of the signal Y-SEL is four times larger than the change of the inputted parameter L. The clock signal CK and the Y-SEL have the same frequency. In this way, for each focal length value, delay parameters can be calculated respectively for four beams.

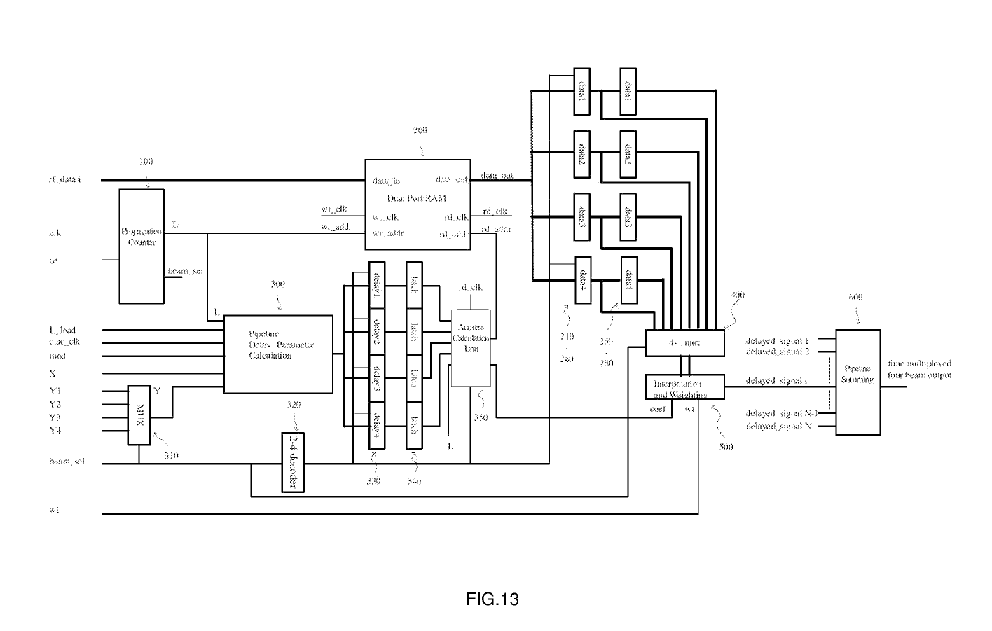

FIG. 13 is a block diagram of an embodiment using the four-beam beamformer shown in FIG. 12. The propagation counter 100 counts sample pulses and record the traveling distance of the ultrasound, and it is thus called as the propagation counter. The output of the propagation counter is used as the write address of the dual port RAM 200. Since the capacity of dual port RAM is needed to accommodate the maximum address difference, the dual port RAM is normally in a range of 256 to 512. The write address and the read address return to the starting point automatically when the counting reaches the end, which is equivalent to a circular storage queue. In this embodiment, the RAM depth is 512, thus it is only necessary to connect the write address of the dual port RAM 200 with the low 9 bit of the propagation counter 100. The echo data rf_data_i is continuously written into the dual port RAM 200 according to the write addresses. As the representation of depth, the output L of the propagation counter is inputted into the delay parameter calculation unit 300. Under the strobe of input pulse L_load, the focal length L is inputted, and the frequency of the pulse L_load is ¼ of calc_clk. That is, for the same focal length L, the delay of the four beams is separately calculated. The input parameter mod of the delay calculation unit is a control command. It consists of two control lines C1 and C2 as shown in FIG. 12. The input parameter X is only related to the channel and will not change along with the beam switching. The beam-related input parameters Y for the four beams are respectively denoted as Y1, Y2, Y3 and Y4, which are switched by the 4-1 multiplexer. The selecting of parameter is controlled by the beam selecting signal beam_sel outputted by the propagation counter 100. During the inputting of parameters Y1-Y4, the delay parameter calculation unit 300 calculates the delay parameters for each beam in pipeline manner, and the delay parameters are inputted into the register group 330, and the registers are represented as delay1-delay4. 2-4 encoder 320 encodes the beam_sel to generate four channels of control output to select the delay1-delay4. The values of the delay1-delay4 are saved in the latch 340 by the falling edge of the finally inputted pulse, in order to maintain the values of the delay1-delay4 unchanged within a focal length change cycle. The values of the delay1-delay4 are inputted into the address calculating unit 350 to calculate the corresponding RAM read addresses by cooperating with the focal length L. In a write cycle, the address calculating unit 350 should calculate a read address once for each beam and read a data form the dual port RAM. Therefore, the frequency of the read pulse rd_clk is four times larger than that of the write pulse wr_clk. If the wr_clk is 40 MHz, then the rd_clk is 160 MHz. The data read out from the dual port RAM 200 is inputted into the registers 210 to 240 in turn. For it is necessary for the interpolation, the history data is saved in the registers 250 to 280. The history data saved is not limited to two stages. According to the order of the interpolation arithmetic, a plurality of stages may be kept. For instance, for six order interpolation, six continuous output data is saved. The data read out is outputted to the interpolation and weighting unit 500 by the 4-1 multiplexer 400 in pipeline manner. The interpolation coefficient of the interpolation and weighting unit is supplied by the address calculating unit 350. The weighted data wt may be calculated, or precalculated and stored in an external memory and read out from the external memory during the receiving. For the change of the weighted value does not need to be fast, for example the scan line of 25 cm changes 64 times, the saving of the weighted values will not occupy the memory space seriously. The data from the interpolation and weighting unit 500 is the four-beam echo data that has been delayed and through time division multiplexing. This data is sent to the summing unit 600 in pipeline manner to be summed up along with the outputs of the other N−1 units to finally obtain the four-beam data output of time division multiplexing.

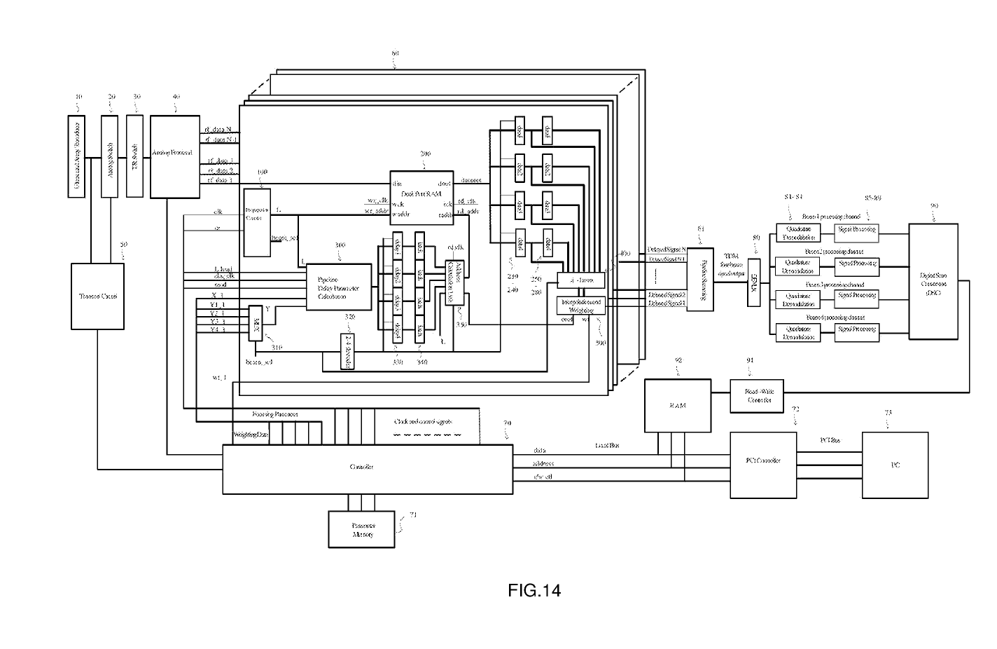

FIG. 14 is a block diagram of a four-beam beamforming B-mode ultrasound system based on real-time delay parameter calculation. The array transducer 10 has 128 array elements. The beamformer 60 has 64 channels in total. Delayed pulse signals are transmitted to a certain group of array elements (called activated array element) by the transmit circuit 50 under the control of the controller 50 so as to realize the focusing transmission. The echo signal of the activated array element is transmitted to the T/R switch 30 after being gated by the analog switch 20. The T/R switch 30 is used for isolating the transmitted high-voltage signal to avoid the subsequent saturation of the amplifying circuit. The analog signal of the T/R switch 30 is transmitted to the analog frontend circuit 40 for amplifying and processing, and the analog frontend comprises a preamplifier, a Time Gain Compensation (TGC) amplifier and an ADC circuit. The amplified signal is converted into digital signal and transmitted to the beamformer 60. The beamformer has a circuit configuration of the 64 channels, as shown in FIG. 13. The 64 channels of input signal are delayed in the beamformer 60. Four delayed beam data are outputted form each channel, and transmitted to the summing unit 61 in time division multiplexing manner. The four delayed beam data are superposed in pipeline manner in the summing unit 61, and the outputs are four beam data of time division multiplexing after beamforming. The time division multiplexed data is divided into four channels after passing through the demultiplexer (DEMUX) 80. Then, they are respectively transmitted to the quadrature demodulators 81 and 84 and the signal processing units 85 and 89. Four scan lines that are formed finally are transmitted to the digital scan converter (DSC) 90. The digital scan converter 90 converts the scan line data into the raster data having rectangular coordinates, and transmitted to the image buffer storage 92 by the read-write controller 91. Under the control of the controller 70, the image data is read and displayed by the computer 73 via PCI bus. The control data is also downlinked to the controller 70 via the PCI bus. The parameters for use in the focusing delay calculation are stored in the data memory 71. Before the starting of each scanning, the controller 70 transmits all the parameters to the beamforming channels and sends out a control sequence to control the proceeding of beamforming. Taken the FIG. 14 as an example, the frame rate of the B-mode ultrasound system is improved by four times with the condition that the image lines density is not reduced. This improves the imaging quality of B-mode ultrasound system with respect to the organ of locomotion such as the heart.

The technical solutions of the present invention are operated in pipeline manner, and by flexible mode control and parameter configuration, the delay calculation unit can be used for the convex array probe, the linear array probe, and the phased array probe.

It should be emphasized that the above-described embodiments of the present invention, particularly, any preferred embodiments, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein within the scope of this disclosure and the present invention and protected by the following claims.

What is claimed is:

1. A multi-beamforming method based on real-time calculation of delay parameter and pipeline technique, wherein at least one multi-beamforming apparatus is used, each multi-beamforming apparatus receives a channel ultrasound echo, each channel ultrasound echo has M number of wave beams with different angles or scanning positions, the method comprises the following steps:
A. each multi-beamforming apparatus processes one channel echo signal and generates delayed signals for M number of scan lines with different scan angles or positions;
B. the echo signal is linearly written into a dual-port or tri-port RAM by each multi-beamforming apparatus;
C. delay parameters for the M number of wave beams in the same channel are calculated by time-division calculation according to array element parameters related to current channel and focus position parameters, and converted into read addresses of the RAM;
D. within one write cycle, echo data is read out from the RAM in turn according to the read addresses to generate M number of delayed signal outputs;
E. the M number of delayed signal outputs generated in the same channel are transmitted to an interpolation unit and a weighing unit in time division manner for performing fine delay and apodization operation;
F. first beams generated by all multi-beamforming apparatus are superposed to obtain a first synthesized beam,
second beams generated by all multi-beamforming apparatus are superposed to obtain a second synthesized beam,
. . .
Mth beams generated by all multi-beamforming apparatus are superposed to obtain a Mth synthesized beam,
the above operations are done in the same summing unit in time-division and pipeline manner;
G. the outputted M number of beam signals are transferred to the next stage summing unit in time division multiplexing manner to perform the final summing between chips or performed with signal processing and image processing.

2. The multi-beamforming method of claim 1, wherein the delay parameter calculation formula in the step C is as follows:
the delay is expressed as:
$${τ}_{i} = \frac{( L - {𝓁}_{i} )}{c}$$
wherein c is sound speed that is about 1540 m/s in the human tissue;
convert the delay into sampling pulse unit:
$${n}_{i} = \frac{( L - {𝓁}_{i} )}{c} {F}_{s}$$
for convex array probe:
lᵢ=\sqrt{(R+L)²+R²−2(R+L)R cos(θᵢ−θᵣ)}
for linear array probe:
lᵢ=\sqrt{L²+(xᵢ−xᵣ)²}
for phased array probe:
lᵢ=\sqrt{xᵢ²+L²−2xᵢL cos(90°−θᵣ)}
wherein, τᵢ represents the delay parameter; nᵢ represents the delay parameter of sampling pulse unit; Fₛ represents the sampling frequency; θᵣ represents the angle between the receive line and the transmit line; polar coordinates of the ith array element is (θᵢ,R); the focus on the receive line is F, and the focal length is L; lᵢ is the sound path from the array element i to the focus F; xᵢ is the coordinates of the ith array element; xᵣ is the coordinates of the scanning line.

3. The multi-beamforming method of claim 2, wherein parameters to be inputted in the present invention are as follows:
a) parameters related to the positions of the probe and the array element in the aperture, that is, the parameters related to the channel, and represented by X here;
b) parameters related to the orientation angle of the scanning line (for convex array probe, phased array probe) or the position of the scanning line (for linear array probe), that is, the parameters related to the wave beam, and represented by Y here;
c) parameters needed to be processed in real time, that is, the focal length of the focus, and represented by L here;
wherein delay calculation system is divided into two portions, common calculation portion and beam related calculation portion; the parameters X related to the channel and the real-time parameter L are sent to common calculation portion; based on the calculation output of the common calculation portion, the parameters related to the beam are calculated according to the inputted parameters Y that are related to the beam.

4. The multi-beamforming method of claim 1, wherein the RAM is a dual-port RAM or a tri-port RAM, the read frequency of the RAM is M times larger than the write frequency.

5. A multi-beamforming apparatus based on real-time calculation of delay parameter and pipeline technique, comprising: an analog frontend that receives echo signal, the analog frontend being connected in sequence to an analog-to-digital conversion module, a DC cancellation module, a write control module, a dual-port or tri-port RAM, an interpolation and weighting unit in pipeline manner and a summing unit in pipeline manner; wherein the multi-beamforming system further comprises a real-time calculation unit of delay parameter in pipeline manner, the real-time calculation unit receives inputted parameters of M number of beams and calculates corresponding delay parameters that are then converted into read addresses of the RAM, the real-time calculation unit is connected in sequence to an address calculation unit, a propagation counter and a read control module, the read control module is connected to the RAM and the interpolation and weighting unit in pipeline manner to control the reading out of the echo signal.

6. The multi-beamforming apparatus of claim 5, wherein the real-time calculation unit of the delay parameter in pipeline manner consists of a common calculation portion A and a beam-related calculation portion B; the portion A processes the channel-related parameter X and the real-time parameter L; the portion B calculates the beam-related parameters according to the inputted beam-related parameters Y, based on the calculation output of the portion A;
the real-time calculation unit of delay parameter in pipeline manner has control signals C1 and C2, and the inputted parameters X,Y are set according to the type of probe, as shown in the following table:

| probe | X | Y | C1 | C2 |
|---|---|---|---|---|
| convex array | R | 2R cos(θᵢ − θᵣ) | 0 | 0 |
| linear array | 0 | (Xi − Xr)² | 1 | 1 |
| phased array | Xᵢ | 2Xᵢ cos(90° − θᵣ) | 0 | 1 |

7. The multi-beamforming apparatus of claim 5, wherein the RAM is a dual-port RAM, the read frequency of the dual port RAM is M times larger than the write frequency; in the multi-beamforming system where the number of beams is M, corresponding to each write data, the real-time address calculating unit will calculate M number of corresponding read addresses according to M number of outputs of the real-time delay parameter calculation unit and read M number of data according to the addresses from the dual port RAM.

8. The multi-beamforming apparatus of claim 5, wherein the RAM is a tri-port RAM that has a write port and two read ports, the read frequency of the tri-port RAM is M times larger than the write frequency; in the multi-beamforming system where the number of beams is 2×M, corresponding to each write data, the real-time address calculating unit will calculate 2×M number of corresponding read addresses according to 2×M number of outputs of the real-time delay parameter calculation unit and read M number of data respectively from each of the two read ports of the tri-port RAM to obtain 2×M number of delay signal outputs.
